(19) United States
(12) Patent Application Publication
(10) Pub. No.: US 2022/0299497 A1
(43) Pub. Date: Sep. 22, 2022

(54) SYSTEM INCLUDING A BOX AND AN INSTRUMENTED CONTAINER FOR DETECTING THE PRESENCE OF MICRO-ORGANISMS IN A LIQUID SAMPLE

(71) Applicant: COMMISSARIAT Á L'ENERGIE ATOMIQUE ET AUX ENERGIES ALTERNATIVES, Paris (FR)

(72) Inventors: Maxime GOUGIS, Grenoble Cedex 9 (FR); Pierre MARCOUX, Grenoble Cedex 9 (FR)

(73) Assignee: COMMISSARIAT Á L'ENERGIE ATOMIQUE ET AUX ENERGIES ALTERNATIVES, Paris (FR)

(21) Appl. No.: 17/636,756

(22) PCT Filed: Aug. 17, 2020
(86) PCT No.: PCT/EP2020/072947

§ 371 Date: Feb. 18, 2022

(30) Foreign Application Priority Data
Aug. 22, 2019 (FR) FR1909333

(51) Int. Cl. G01N 33/49; C12Q 1/04

(52) U.S. Cl. CPC G01N 33/49; C12Q 1/04; G01N 27/4035

(57) ABSTRACT

A system for detecting the presence of microorganisms present in a sample includes an enclosure having a closed internal space bounded by a receptacle and a lid configured to be affixed to the receptacle to close the enclosure hermetically, an electronic unit for acquiring pH-measurement data integrated into the enclosure, a heating unit integrated into the enclosure for heating the internal space, a unit for measuring and managing temperature having a temperature-measuring probe placed inside the enclosure, and a processing unit connected to the electronic unit and configured to process measurement data. The processing unit includes a module for detecting the presence of microorganisms via analysis of variation in the pH measured in a liquid sample, and an instrumented container housed in the internal space of the receptacle for receiving the sample. The container includes reference and pH-measuring electrodes that are integrated into walls of the container.

### TECHNICAL FIELD OF THE INVENTION

The present invention relates to a system notably comprising an enclosure in which an instrumented container is placed, and to a method for detecting the presence of microorganisms in a liquid sample placed in the container of the system. Detection may notably be achieved electrochemically.

### PRIOR ART

Blood is normally completely sterile. Any microorganism present in the blood therefore represents a threat to the health of the human body. Blood culture is currently the only method for diagnosing bloodstream infections. It is a test frequently used in clinical microbiology. A blood culture firstly consists in culturing the blood in a nutrient broth under aerobic and anaerobic conditions, under incubation at 37° C. in automatic devices suitable for this type of sample, in order to obtain bacterial growth. The objective is to amplify the amount of bacteria present in the sample, by providing it with favorable growth conditions, and thus to detect the presence of bacteria in the blood of a patient presenting sepsis. This amplification is especially necessary since bacterial concentration in blood during bacteremia is always low, of the order of one or a few bacteria per milliliter of blood sampled. This first step of the blood culture therefore consists in a simple detection of presence, without identification, with respect to a normal flora-free sample: the presence of any bacteria or fungus therefore leads to a positive test.

This detection will then be followed by subculture on an agar plate with a view to isolating the pathogen in colonies, allowing the pathogen and its antibiogram to then be identified. In the case of bloodstream infections, examinations are urgent and any delay in obtaining results (positivity of the blood culture, identity of the pathogen, antibiogram) has an undeniable impact on the course of treatment of the patient (mortality, length of hospitalization, complications, etc.). Specifically, the results of blood cultures allow the antibiotic therapy employed to be tailored to the specific case of the patient: the earlier a tailored and effective antibiotic therapy is provided, the greater the patient's chances of survival. Each hour of delay in initiating a tailored antibiotic therapy is associated with an increase in mortality.

The manufacturers of diagnostic tools have worked for many years to decrease the time required to determine whether a blood culture is positive, and to decrease the analysis times of identification and antibiogram tests. Currently, two main types of automatic device coexist:
Devices that monitor the amount of carbonic acid generated in the liquid phase, by virtue of a polymer (silicone) matrix loaded with a pH-sensitive chromophore or fluorophore.
Devices that detect an increase in total pressure in the gas phase.

These two technologies have in common that the automatic devices used to implement them are large in size, and that it is impossible to start testing until the vial is in the automatic device (pre-incubation generates false negatives); hence precious time is lost before an optimized antibiotic therapy is delivered. A study in 2013 showed that average transport time was 9 hours (interquartile range: 3-15 hours), with 6% of vials having a transport time longer than 20 hours.

There is therefore a need to provide a solution that:
allows the time between the moment at which the sample is taken and the start of the analysis of the sample to be decreased;
is easily transportable, so as to be available in the field;
is easy to implement, even by personnel who are not highly qualified.

### SUMMARY OF THE INVENTION

This need is met by a system for detecting the presence of microorganisms present in a sample, this system comprising:
an enclosure comprising a closed internal space bounded by a receptacle and a lid that is intended to be affixed to the receptacle to close the enclosure hermetically,
an electronic unit for acquiring pH-measurement data, which is integrated into said enclosure,
two second electrical contacts that are connected to the electronic acquiring unit and arranged inside the enclosure,
a heating unit integrated into the enclosure with a view to heating the internal space of the enclosure,
a unit for measuring and managing temperature, comprising a temperature-measuring probe placed inside the enclosure,
an electrical power source for powering the electronic acquiring unit and the heating unit,
a processing unit connected to said electronic acquiring unit and configured to process the measurement data and comprising a module for detecting the presence of microorganisms via analysis of the variation in the pH measured in the liquid sample,
an instrumented container housed in the internal space of the receptacle and intended to receive said sample, said container comprising:
   walls comprising a first surface, called the internal surface, which makes contact with the internal volume thereof intended to receive said sample, and a surface, called the external surface, opposite said internal surface,
   a reference electrode and a pH-measuring electrode that are integrated into at least a first wall of said walls of the container and that each comprise at least two conductive portions that are connected together, a first portion arranged on the internal surface and intended to make contact with the liquid sample placed inside the container, and a second portion arranged on the external surface and comprising two separate first electrical contacts that are accessible from outside the container, its first electrical contacts each bearing against a separate second electrical contact of said enclosure.

According to one particularity, the second electrical contacts take the form of two conductive rings arranged concentrically.

According to another particularity, the electronic acquiring unit comprises a first wireless communication module and the processing unit comprises a second wireless communication module linked to the first wireless communication module via a wireless communication link.

According to another particularity, the system comprises a unit for agitating by vibration integrated into said receptacle.

According to another particularity, the processing unit is integrated into said enclosure.

According to another particularity, the first portion of the reference electrode and the first portion of the measuring electrode each take the form of a pad flush with the internal surface of said first wall.

According to another particularity, the two pads each take the form of a deposit of a conductive ink screen-printed on the internal surface of said first wall.

According to another particularity, the pad of the measuring electrode is made of a material chosen from iridium oxide and a conductive polymer.

According to another particularity, the pad of the reference electrode is of Ag/AgCl type.

According to another particularity, the two first electrical contacts are integrated into said first wall so as to each form one contact land or two concentric rings.

The invention also relates to a method for detecting the presence of microorganisms in a liquid sample, implemented using the system such as defined above, said method comprising the following steps:

introducing the liquid sample and a culture medium into the container, to obtain a liquid mixture,

introducing said container into the internal space of the enclosure,

adjusting the incubation temperature in the internal space of the enclosure to a set value suitable for the culture medium,

measuring pH over time in the liquid mixture present in the container, to obtain a curve of pH variation as a function of time,

determining the presence or absence of microorganisms via analysis of said curve of pH variation as a function of time.

According to one particularity, the method comprises a step of agitating the container to mix the liquid sample and the culture medium.

According to another particularity, the method comprises a step of adjusting the incubation time.

The solution of the invention thus allows each container to be equipped with its own instrument for monitoring microbial growth, this allowing the blood-culture test to be started outside the analysis laboratory, at the very place of sampling.

### BRIEF DESCRIPTION OF THE FIGURES

Other features and advantages will become apparent from the following detailed description, which is provided with reference to the appended drawings, in which:

FIG. 1A shows a vial-type instrumented container according to that used in the system of the invention.

FIG. 1B shows, viewed from above, the bottom of said vial and shows the two electrodes integrated into the vial.

FIG. 2A schematically shows the system for detecting the presence of microorganisms according to the invention.

FIG. 2B shows, viewed from above, the principle of production of the electrical contacts used in the enclosure of the system of the invention.

FIG. 3 shows a chart presenting the various steps of the method of the invention implemented using the system of FIG. 2.

FIG. 4 shows two curves of pH variation as a function of time, obtained by virtue of the system according to the invention.

### DETAILED DESCRIPTION OF AT LEAST ONE EMBODIMENT

In the remainder of the description, the term “integrate” is to be understood to mean forming a part, of a given assembly, that is non-separable without the use of suitable tools.

The proposed invention notably allows the time between the moment at which a sample is taken and its analysis to be considerably decreased.

The invention consists, inter alia, in providing a stand-alone and unitary automatic device for detecting the presence of microorganisms in a liquid sample, via electrochemical measurement of pH.

In particular, the system is suitable for detecting the presence of a pathogen in a blood sample, via electrochemical measurement of pH during a blood culture.

However, it may also be adapted to other biological fluids, such as, for example, joint fluids, pleural fluids, pericardial fluids and other puncture fluids, vitreous fluids and pus.

Also in the clinical field, it may be used to test the sterility of solid objects such as biopsies. At the industrial level, such a system allows sterility tests to be performed on finished products (vials, syringes, needles, prostheses, parenteral nutrition bags, contact lenses, injectable drugs, etc.), or raw materials (active ingredients, excipients, cosmetic products, etc.). The object to be tested is then submerged in an agar or liquid nutrient medium, then monitored during incubation (at 30° C. or 37° C.) to see whether microbial growth occurs in the nutrient medium. When the test is performed with an agar nutrient medium, a Petri dish will preferably be used, in which case the system is applied not to a vial but to a Petri dish.

Since detection must be non-specific, it is important to base it on a particularly generic indicator: the emission of CO₂ is one such parameter since this metabolite is common to all microorganisms. Specifically, the high metabolism of bacteria (of the order of one W/g) is accompanied by a substantial emission of CO₂, of the order of 700 000 molecules per second and per bacterium during the exponential phase of growth. It is this effect that the system of the invention makes it possible to take advantage of, by detecting the acidification that attends the emission of CO₂. In the case of a blood sample, the invention therefore notably aims to be able to detect a pathogen in a blood culture by virtue of electrodes that are used to monitor pH electrochemically. Using electrochemistry allows the blood culture to be monitored with a low-cost, compact, disposable, sterilizable sensor that consumes little power and that may be manufactured on a large scale. Likewise, in the case of sterility tests, the invention aims to detect the acidification, of the liquid or agar nutrient medium, that attends microbial growth if the tested object is not sterile.

This principle is notably applied using a container that may for example be an instrumented vial 1 intended to receive the liquid sample ECH taken or a Petri dish as mentioned above.

In the rest of the description, the invention is described in the case of a vial 1, but it may be adapted to any type of container.

The vial is a unitary element, advantageously made of disposable and optionally biosourced plastic.

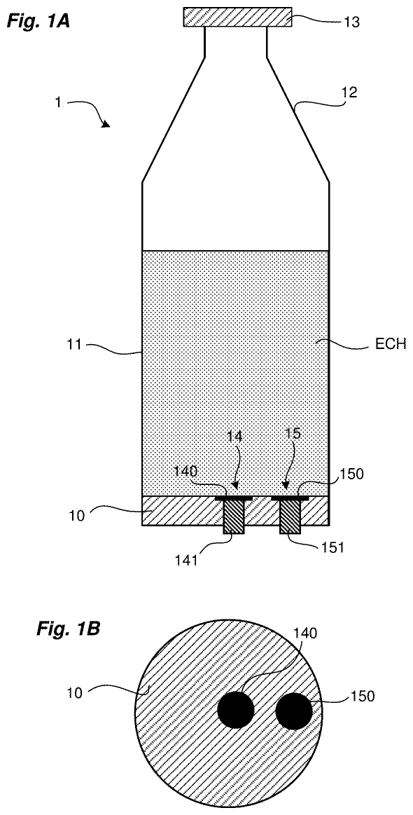

With reference to FIG. 1A, this vial 1 may have a conventional shape with a bottom wall 10 forming the bottom of the vial, with a side wall 11 forming the body and with a neck 12 in its top portion, the neck ending in the mouth. The walls of the vial bound the internal volume of the vial. The walls of the vial have an internal surface located inside the vial and an external surface located outside the vial. The vial also comprises a stopper 13 that is affixed to its mouth to close it hermetically.

According to one particularity of the invention, two electrodes 14, 15, namely a measuring electrode 14 and a reference electrode 15, are integrated into the vial 1. The two electrodes are advantageously integrated into the bottom wall 10 of the vial 1. The electrodes 14, 15 may be covered with a protective layer in order to improve the stability over time of the vial and notably to protect the electrodes from possible fouling or biofouling, i.e. saturation of the surface of the electrodes with proteins, lipids or cells.

Each electrode 14, 15 may be made of two separate conductive portions:
a first portion arranged on the internal surface of a wall of the vial 1, the bottom wall 10 for example, in the internal volume of the vial, to make contact with the liquid sample to be analyzed, which is placed in the vial;
a second portion connected to the first portion through a wall of the vial, the bottom wall 10 for example, and arranged on the external surface of the vial so as to form an electrical contact 141, 151 to be connected.

The first portion of each electrode may take the form of a pad 140, 150 (FIG. 1B). The pad may be housed in a cavity of the bottom wall 10 and be flush with the internal surface of the vial.

The two pads may each take the form of a deposit of a conductive ink screen-printed on the internal surface of the bottom wall.

The first portion of the measuring electrode 14 may be made of a material chosen from iridium oxide, which is an oxide the redox potential of which depends on pH, and a conductive polymer, such as polyaniline (PAni).

The first portion of the reference electrode 15 may be of Ag/AgCl type, this type of electrode conventionally being used in electrochemistry.

The two electrical contacts 141, 151 of the vial are integrated into said first wall in such a way that each forms one contact land. A variant of embodiment of the electrical contacts will be described below.

The vial 1 may bear an identification tag, an RFID tag for example, in which data related to the sample are stored (name of the patient, amount sampled, date and time at which the sample was taken, etc.). It will be seen that this tag may be read by the processing unit UC of the system and its data stored in memory by the processing unit. The processing unit UC will then be able to directly correlate the results of the test with the sample data stored in memory beforehand.

In addition to the vial 1 thus equipped, the system comprises an enclosure 2 intended to receive the vial for analysis.

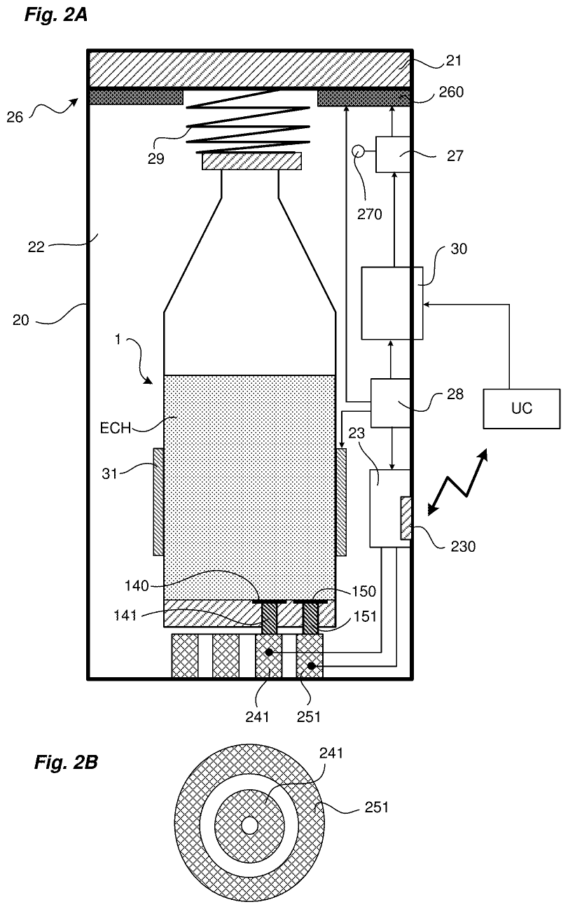

With reference to FIG. 2A, the enclosure 2 may be composed of a receptacle and of a lid 21 that is intended to be affixed to the receptacle 20 to close the enclosure hermetically. The enclosure has properties with respect to thermal insulation that allow its internal space to be thermally insulated from the exterior. A double-walled solution may be employed.

The enclosure 2 thus comprises an internal space 22 bounded by the walls of its receptacle 20 and of its lid 21.

The system also comprises:
an electronic unit 23 for acquiring pH-measurement data, which is integrated into said enclosure,
two other electrical contacts 241, 251 that are electrically connected to the electronic acquiring unit 23 and arranged inside the enclosure 2,
a heating unit 26 integrated into the enclosure with a view to heating the internal space of the enclosure,
a unit 27 for measuring and managing temperature, notably comprising a temperature-measuring probe 270 placed in the internal space 22 of the enclosure 2,
an electrical power source 28 for powering the electronic acquiring unit 23 and the heating unit 26.

The acquiring unit 23 may be a conventional circuit board configured to measure the electric potential between the two electrodes 14, 15 of the vial 1. The measurement data are stored on the board. The board may comprise a communication module 230. The communication module 230 may be of the wireless type and operate under a protocol such as Bluetooth or equivalent.

As shown in FIG. 2B, the two electrical contacts 241, 251 of the enclosure 2 may take the form of two conductive metal rings arranged concentrically. They are advantageously positioned inside the enclosure, at the bottom thereof and in a manner suitable for connecting to the two contact lands of the vial 1 when the latter is inserted into the enclosure. Providing two ring-shaped contacts makes it possible for the vial 1 to be connected 360° around its axis when it is inserted in the enclosure 2.

Alternatively, the two concentric ring-shaped contacts could be borne by the vial 1 and the contact lands by the enclosure 2. It would also be possible to provide concentric ring-shaped electrical contacts both on the vial 1 and on the enclosure 2.

The vial 1 is intended to be placed in the internal space of the enclosure. The enclosure 2 and the vial 1 may interact with each other to ensure a contact pressure is exerted between the electrical contacts 141, 151 of the vial and the electrical contacts 241, 251 of the enclosure. A spring 29 may be attached to the lid 21 and bear against the vial 1 when the lid 21 is closed on the receptacle 20, so as to press the vial against the bottom of the enclosure 2. Any other solution may of course be envisaged.

The heating unit 26 is of electric type and may comprise a resistive heater placed in the internal space of the enclosure or a solution employing a thermoelectric module. The heating must generate heat uniformly, all around the vial. Non-limitingly, in FIG. 2A, the resistor 260 is positioned under the lid 21. Contact may be made between the resistor and the electrical power source 28 when the lid 21 is closed on the receptacle 20. A mechanism employing sliding contacts may be provided.

The unit 27 for measuring and managing temperature comprises a temperature probe 270 and means for regulating said temperature to a determined set value. The temperature value is chosen depending on the type of culture employed. In the case of a blood culture, the incubation temperature is set equal to 35° C.+/−2° C.

Apart from blood, a sterility test may be performed for other liquid samples. Specifically, it is possible to place other biological fluids in the vial, for example joint fluids, pleural fluids, pericardial fluids and other puncture fluids, vitreous fluids and pus. The incubation temperature will also be 35° C.+/−2° C. For sterility tests, the incubation temperature will be 30-35° C. when the medium is a thioglycolate broth and 20-25° C. when the medium is a casein-soya broth.

The unit 27 for measuring and managing temperature may comprise a thermostat intended to receive the measured temperature value and to control the heating unit 26 to regulate the temperature to the desired value.

The system may also comprise a human-machine interface 30. This interface may be composed of a screen arranged on the casing of the enclosure 2 and of control members, notably intended to allow the temperature to which the inside of the enclosure is heated to be adjusted. This interface may also comprise luminous indicators providing information on the operating status of the system and on the status of the detection (an indicator light may turn on in the event of detection of the presence of bacteria).

The electrical power source 28 may consist of a rechargeable battery housed in a compartment of the enclosure. It is intended to power the heating unit 26, the unit 27 for measuring and managing temperature, the unit 23 for acquiring measurement data, and the human-machine interface 30 if the latter is present.

As already specified above, the system comprises a processing unit UC comprising a microprocessor and storage means. It may also comprise a communication module. This module may be wireless, with a view to communication via a wireless link, for example one according to the Bluetooth protocol, with the corresponding module 230 of the acquiring unit 23.

The processing unit UC is configured to:
receive the measurement data from the acquiring unit 23, for example via the wireless link,
establish the curve of pH variation of the mixture as a function of time,
process the obtained curve and determine whether microorganisms are present,
control the human-machine interface 30 attached to the enclosure to indicate the presence of any bacteria,
read the RFID tag present on the vial 1 in order to collect and store the data related to the sample,
ensure the regulation of the heating unit 26 to ensure the most constant possible incubation temperature.

The processing unit UC may consist of a personal computer external to the enclosure 2 or be directly integrated into the enclosure 2, thus allowing a unitary tool that is stand-alone with respect to power and in operation to be obtained. In the latter case, the processing unit would be powered by the power source 28 of the enclosure.

The system may comprise a unit 31 for agitating the vial 1, which unit is placed in the enclosure. This agitating unit is integrated into the enclosure and may notably allow mixing between the liquid sample taken and placed in the vial and the culture medium added to the vial 1 with a view to growing the microorganisms to be promoted. This agitating unit may consist of a vibratory plate or sleeve (as in FIG. 2A) integrated into the enclosure 2, and against which the vial will bear, or of any other equivalent solution. This agitating unit may also be a rotating magnet (magnet placed on a motor), so as to rotate a disposable magnetic bar placed in the sample (magnetic stirring). The agitating unit 31 is advantageously powered by the power source 28.

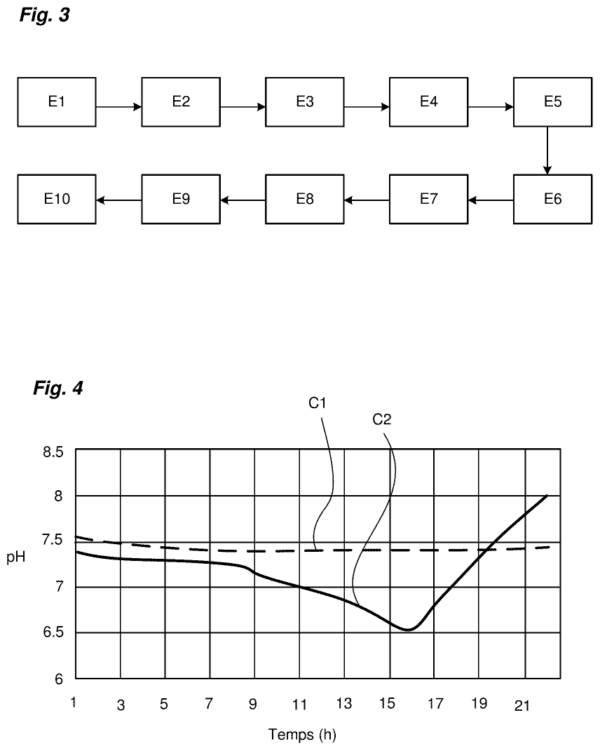

With reference to FIG. 3, after taking a sample from a patient, for example with a view to performing a sterility test, the operating principle of the system may notably be as follows:
E1: Introducing the liquid sample into the vial 1, which already contains a culture medium suitable for the culture of microorganisms.
E2: Optionally, having the processing unit UC read the RFID tag, in order to collect the data related to the taken sample.
E3: Introducing the vial 1 thus filled into the receptacle 20 and closing the lid 21 with a view to ensuring the electrical contact by virtue of the spring 29. Stops and poka-yokes may allow the vial 1 to be wedged in the receptacle 20 and ensure the electrical connection between the electrical contacts 141, 151 of the vial and the electrical contacts 241, 251 of the enclosure.
E4: Adjusting the temperature in the internal space of the enclosure to a set value suitable for the culture medium. The adjustment may be carried out directly via the human-machine interface 30 integrated into the enclosure 2. The vial must be heated uniformly to a set and regulated temperature, to promote the growth of pathogens and their detection. Furthermore, it may also be possible to adjust incubation time. Conventionally, incubation lasts 5 days but may sometimes be extended to 15 days in certain situations (for example: suspicion of endocarditis) or for certain samples (for example: joint fluids). Sterility tests generally take longer (up to 3 weeks of incubation before it is possible to deliver a negative result) because the contaminating microorganisms may be fastidious and/or very stressed.
E5: Advantageously, activating the agitating unit 31 to promote mixing between the liquid sample and the culture medium. This also shortens microorganism detection time by ensuring homogenization of the culture medium during incubation, and oxygenation of this medium.
E6: Measuring the pH in the liquid mixture present in the vial 1 and acquiring pH-measurement data over time with the acquiring unit 23.
E7: Transferring the measurement data obtained by the acquiring unit 23 to the processing unit UC, for example via the wireless communication link.
E8: Establishing, with the processing unit UC, a curve of pH variation as a function of time.
E9: Analyzing the curve obtained by the processing unit UC and determining whether microorganisms are present or absent via analysis of said curve of pH variation as a function of time.
E10: In the event of detection of the presence of bacteria, controlling the human-machine interface 30 with the processing unit 23 to flag this status.

By way of example, FIG. 4 shows two curves of pH variation obtained during a blood culture implemented using the system of the invention. The first curve C1 was obtained for a control sample, without bacteria, and the second curve C2 was obtained for a sample containing bacteria (in an initial concentration of 10 cfu/ml).

In curve C2, a downward variation in pH after 8 hours, synonymous with the presence of bacteria in the analyzed sample, may notably be seen.

The pH variation may for example be detected using a threshold applied to the first derivative of the pH curve. Of course, any other solution could be used to process the curve. In all cases, it is a question of detecting a drop in pH, synonymous with an increase in the acidity of the aqueous medium following the emission of CO₂ by microorganisms.

In order to make the measurement more reliable, it would be possible to integrate a plurality of pH sensors into the system.

1. A system for detecting the presence of microorganisms present in a sample, comprising:
an enclosure including a closed internal space bounded by a receptacle and a lid that is configured to be affixed to the receptacle to close the enclosure hermetically,
an electronic unit configured to acquire pH-measurement data, integrated into the enclosure,
two second electrical contacts connected to the electronic acquiring unit and arranged inside the enclosure,
a heating unit integrated into the enclosure configured to heat the internal space of the enclosure,
a unit for measuring and managing temperature, comprising a temperature-measuring probe is placed inside the enclosure,
an electrical power source configured to power the electronic acquiring unit and the heating unit,
a processing unit connected to the electronic acquiring unit and configured to process the measurement data and comprising a module for detecting the presence of microorganisms via analysis of variation in the pH measured in a liquid sample, and
an instrumented container housed in the internal space of the receptacle and configured to receive the sample, the container comprising:
walls comprising an internal surface, which makes contact with an internal volume of the container and configured to receive the sample, and an external surface, opposite the internal surface, and
a reference electrode and a pH-measuring electrode that are integrated into at least a first wall of the walls of the container and that each comprise at least two conductive portions that are connected together, a first portion arranged on the internal surface and configured to make contact with the liquid sample placed inside the container, and a second portion arranged on the external surface and comprising two separate first electrical contacts that are accessible from outside of the container, the first electrical contacts each bearing against one of the second electrical contacts of the enclosure.

2. The system as claimed in claim 1, wherein the second electrical contacts comprise two conductive rings arranged concentrically.

3. The system as claimed in claim 1, wherein the electronic acquiring unit comprises a first wireless communication module and the processing unit comprises a second wireless communication module linked to the first wireless communication module via a wireless communication link.

4. The system as claimed in claim 1, wherein comprising a unit for agitating by vibration integrated into the receptacle.

5. The system as claimed in claim 1, wherein the processing unit is integrated into the enclosure (2).

6. The system as claimed in claim 1, wherein the first portion of the reference electrode and the first portion of the measuring electrode each comprise a pad flush with the internal surface of said first wall.

7. The system as claimed in claim 6, wherein the two pads each comprise a deposit of a conductive ink screen-printed on the internal surface of the first wall.

8. The system as claimed in claim 7, wherein the pad of the measuring electrode is made of a material chosen from iridium oxide and a conductive polymer.

9. The system as claimed in claim 7, wherein the pad of the reference electrode is of Ag/AgCl type.

10. The system as claimed in claim 1, wherein the two first electrical contacts are integrated into the first wall so as to each form one contact land or two concentric rings.

11. A method for detecting the presence of microorganisms in a liquid sample, implemented using the system such as defined in claim 1, comprising:
introducing the liquid sample and a culture medium into the container, to obtain a liquid mixture,
introducing the container into the internal space of the enclosure,
adjusting an incubation temperature in the internal space of the enclosure to a set value suitable for the culture medium,
measuring pH over time in the liquid mixture present in the container, to obtain a curve of pH variation as a function of time, and
determining the presence or absence of microorganisms via analysis of the curve of pH variation as a function of time.

12. The method as claimed in claim 11, comprising agitating the container to mix the liquid sample and the culture medium.

13. The method as claimed in claim 11, comprising adjusting an incubation time.
